(19) United States
(12) Patent Application Publication
(10) Pub. No.: US 2005/0084962 A1
(43) Pub. Date: Apr. 21, 2005

(54) Methods of treatment using electromagnetic field stimulated stem cells

(76) Inventor: Bruce Simon, Mountain Lakes, NJ (US)

Correspondence Address: HARNESS, DICKEY & PIERCE, P.L.C., P.O. BOX 828, BLOOMFIELD HILLS, MI, 48303, US

(21) Appl. No.: 10/924,241

(22) Filed: Aug. 20, 2004

Related U.S. Application Data
(60) Provisional application No. 60/496,526, filed on Aug. 20, 2003.

(51) Int. Cl. C12N 5/08; C12N 15/01

(52) U.S. Cl. 435/366; 435/446

(57) ABSTRACT

Methods of modifying stem cells, in particular mesenchymal stem cells, using electric or electromagnetic fields. In one embodiment, the present invention provides methods of modulating a mesenchymal stem cell activity, the method comprising administering electric stimulation to mesenchymal stem cells in vitro. In another embodiment, the present invention provides methods for the treatment of a human or other mammal subject in need thereof, comprising providing an in vitro culture comprising mesenchymal stem cells, administering an electric stimulation to the in vitro culture, and implanting the mesenchymal stem cells into the mammal subject. In another embodiment, the present invention provides methods for the treatment of a human or other mammal subject, the method comprising implanting mesenchymal stem cells into the mammal subject, and administering an electric stimulation to the mesenchymal stem cells in situ. The present invention also comprises compositions comprising mesenchymal stem cells treated with electric stimulation.

This application claims the benefit of U.S. Provisional Application No. 60/496,526, filed on Aug. 20, 2003.

### INTRODUCTION

The present invention relates to methods and compositions for treating tissue disorders using modified stem cells. In particular, the invention relates to such methods using mesenchymal stem cells that have been subjected to electric fields or electromagnetic fields, such as pulsed electromagnetic fields (PEMFs).

Stem cells can be obtained from embryonic or adult tissues of humans or other animals. Irrespective of tissue origin, all stem cells are unspecialized, are capable of dividing and renewal, and can give rise to specialized cell types (see, e.g., National Institutes of Health, Stem cell Basics, available on the internet at http://stemcells.nih.gov/infoCenter/stemCellBasics.asp). For example, mesenchymal stem cells, which are stem cells obtained from adult or embryonic connective tissues, can differentiate into many different cell types, such as, for example, bone, cartilage, fat, ligament, muscle and tendon.

The use of stem cells in certain therapeutic applications has been investigated. See, for example: U.S. Pat. No. 5,197,985, Caplan et al., issued Mar. 30, 1993; U.S. Pat. No. 5,226,914, Caplan et al., issued Mar. 30, 1993; U.S. Pat. No. 5,486,359, Caplan et al., issued Mar. 30, 1993; U.S. Pat. No. 5,811,094, Caplan et al., issued Sep. 22, 1998; U.S. Pat. No. 6,355,239, Bruder et al., issued Mar. 12, 2002; U.S. Pat. No. 6,387,367, Davis-Sproul et al., issued May 14, 2002; U.S. Pat. No. 6,541,024, Kadiyala et al., issued Apr. 1, 2003; and Eppich et al., Nature Biotechnology 18: 882-887, 2000. However, none of the above references describe exposure of stem cells in vitro to electromagnetic fields to alter beneficially the stem cells for therapeutic use, nor do they disclose implantation of stem cells to a mammalian recipient followed by in situ application of electric fields.

Therapies involving the alteration of cell or tissue properties by exposure to electromagnetic fields has been also been proposed. See, Aaron and Ciombor, J. Cellular Biochemistry 52: 42-46, 1993; Aaron et al., J. Bone Miner. Res. 4: 227-233, 1989; Aaron and Ciombor, J. Orthop. Res. 14: 582-589, 1996; Aaron et al., Bioelectromagnetics 20: 453-458, 1999; Aaron et al, J. Orthop. Res. 20: 233-240, 2000; Ciambor et al., J. Orthop. Res. 20: 40-50, 2000; U.S. Pat. No. 6,485,963, Wolf et al., issued Nov. 26, 2002; U.S. Pat. No. 5,292,252, Nickerson et al., issued Mar. 8, 1994; U.S. Pat. No. 6,235,251, Davidson, issued May 22, 2001; and Binderman et al., Biochimica et Biophysica Acta 844: 273-279, 1985. However, these references do not suggest treatment of mesenchymal stem cells with an electromagnetic field in vitro prior to transplantation to a mammalian recipient, nor do they describe implantation of mesenchymal stem cells to a recipient mammal followed by in situ exposure of the cells to an electromagnetic field.

### SUMMARY

The present invention provides methods of modifying stem cells using electric or electromagnetic fields. In one embodiment, the present invention provides a method of increasing proliferation rate of mesenchymal stem cells, comprising administering electric stimulation to mesenchymal stem cells in vitro. In another embodiment, the present invention provides a method of promoting differentiation of mesenchymal stem cells, comprising administering electric stimulation to mesenchymal stem cells in vitro. In another embodiment, the present invention provides methods for the treatment of a human or other mammal subject in need thereof, comprising providing an in vitro culture comprising mesenchymal stem cells, administering an electric stimulation to the in vitro culture, and implanting the mesenchymal stem cells into the mammal subject. In another embodiment, the present invention provides methods for the treatment of a human or other mammal subject in need thereof, comprising implanting mesenchymal stem cells into the mammal subject, and administering an electric stimulation to the mesenchymal stem cells in situ. The present invention also provides compositions comprising mesenchymal stem cells treated with electric stimulation.

The present invention affords benefits over methods among those known in the art. Such benefits include one or more of increased or accelerated stem cell proliferation; enhanced control of stem cell differentiation, enhanced maintenance of stem cell differentiation; enhanced ability for use of stem cells in tissue engineering applications; enhanced modulation of stem cell activity after implantation; the ability to effect tissue healing or growth without use of non-autologous growth factors; increased rate of healing of tissue defects; and more complete healing of tissue defects. Further areas of applicability and advantages will become apparent from the following description. It should be understood that the description and specific examples, while exemplifying embodiments of the invention, are intended for purposes of illustration only and are not intended to limit the scope of the invention.

### DESCRIPTION

The following definitions and non-limiting guidelines must be considered in reviewing the description of this invention set forth herein.

The headings (such as “Introduction” and “Summary,”) and sub-headings (such as “Stem Cells”) used herein are intended only for general organization of topics within the disclosure of the invention, and are not intended to limit the disclosure of the invention or any aspect thereof. In particular, subject matter disclosed in the “Introduction” may include aspects of technology within the scope of the invention, and may not constitute a recitation of prior art. Subject matter disclosed in the “Summary” is not an exhaustive or complete disclosure of the entire scope of the invention or any embodiments thereof.

The citation of references herein does not constitute an admission that those references are prior art or have any relevance to the patentability of the invention disclosed herein. Any discussion of the content of references cited in the Introduction is intended merely to provide a general summary of assertions made by the authors of the references, and does not constitute an admission as to the accuracy of the content of such references. All references cited in the Description section of this specification are hereby incorporated by reference in their entirety.

The description and specific examples, while indicating embodiments of the invention, are intended for purposes of illustration only and are not intended to limit the scope of the invention. Moreover, recitation of multiple embodiments having stated features is not intended to exclude other embodiments having additional features, or other embodiments incorporating different combinations of the stated features. Specific Examples are provided for illustrative purposes of how to make, use and practice the compositions and methods of this invention and, unless explicitly stated otherwise, are not intended to be a representation that given embodiments of this invention have, or have not, been made or tested.

As used herein, the words “preferred” and “preferably” refer to embodiments of the invention that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As used herein, the word “include,” and its variants, is intended to be non-limiting, such that recitation of items in a list is not to the exclusion of other like items that may also be useful in the materials, compositions, devices, and methods of this invention.

The present invention involves the treatment of tissue defects in humans or other animal subjects. Specific materials to be used in the invention must, accordingly, be pharmaceutically acceptable and biocompatible. As used herein, such a “pharmaceutically acceptable” component is one that is suitable for use with humans and/or animals without undue adverse side effects (such as toxicity, irritation, and allergic response) commensurate with a reasonable benefit/risk ratio. As used herein, such a “biocompatible” component is one that is suitable for use with humans and/or animals without undue adverse side effects (such as toxicity, irritation, and allergic response) commensurate with a reasonable benefit/risk ratio.

Stem Cells:

The compositions and methods of this method comprise and use stem cells. Preferably the stem cells are mammalian stem cells, preferably human stem cells. Stem cells can be stem cells derived from any known tissue source, for example, cartilage, fat, bone tissue (such as bone marrow), ligament, muscle, synovia, tendon, umbilical cord (such as umbilical cord blood), and embryos. In a preferred embodiment, stem cells are derived from bone tissue.

In a preferred embodiment, the stem cells of the invention are mesenchymal stem cells. For example, the stem cells may be mesenchymal stem cells, and are capable of differentiating into bone, cartilage, vasculature or blood cells. Also, for example, the mesenchymal stem cells can be osteogenic stem cells, chondrogenic stem cells, angiogenic stem cells, or hematopoietic stem cells. In a preferred embodiment, the mesenchymal stem cells are embryonic or adult mesenchymal stem cells derived from embryonic or adult tissues, respectively, wherein “adult stem cells” include stem cells established from any post-embryonic tissue irrespective of donor age.

In one embodiment, the stem cells are autologous stem cells. In another embodiment, the stem cells are allogeneic stem cells. In another embodiment, the stem cells are xenogeneic stem cells. Preferably, the stem cells are autologous stem cells or allogeneic stem cells. More preferably, the stem cells are autologous mesenchymal stem cells or allogeneic mesenchymal stem cells. Most preferably, the stem cells are autologous mesenchymal stem cells.

Stem cells utilized in the present invention can be collected, established into cell lines, and propagated in vitro by methods including standard methods among those known in the art. Such methods include those disclosed in U.S. Pat. No. 6,355,239, Bruder et al., issued Mar. 12, 2002; and U.S. Pat. No. 6,541,024, Kadiyala et al, issued Apr. 1, 2003. The stem cells of this invention additionally can also be grown in vitro in a culture medium comprising one or more growth factors, such as, for example, VEGF-1, a fibroblast growth factor (FGF) such as FGF-2, epidermal growth factor (EGF), an insulin-like growth factor-1 (IGF) such as IGF-1 or IGF-II, a tranforming growth factor (TGF) such as TGF-β, platelet-derived growth factor (PDGF), EGM, and/or a bone morphogenetic protein (BMP) such as BMP-2, BMP-4, BMP-6 or BMP-7.

The present invention provides methods of modulating a stem cell activity, comprising administering electric stimulation to stem cells. As referred to herein, “modulating” refers to the modification of one or more activities of stem cells by, for example, enhancing or increasing such activities. In one embodiment, the activity is one or more of increased proliferation, enhanced production of molecules normally produced by the stem cells (such as molecular components of the extracellular matrix, ECM), and accelerated differentiation of stem cells into differentiated cell types. Accelerated differentiation of stem cells includes enhanced production of differentiation markers of differentiated cell types derived from the stem cells, such as, for example, specialized ECM markers, wherein “enhanced production” includes increased and/or accelerated production of such markers, as compared to control stem cells that are not subjected to an electric or electromagnetic field but are otherwise under the same conditions.

Electric Stimulation:

The term “electric stimulation” as used herein includes exposing stem cells to an electric field, such as a direct current electric field, a capacitatively coupled electric field, an electromagnetic field, or combinations thereof. The term “electric stimulation field,” as used herein, does not include an electric or electromagnetic field associated with ambient conditions, such as, for example, an electric field generated by a desktop computer. Electric stimulation comprises exposing stem cells to an electric or electromagnetic field in vitro, or in situ after implantation into a human or other animal subject in need thereof. In one embodiment, the electromagnetic field is a pulsed electromagnetic field (PEMF). In one embodiment, the stem cells are exposed to electric stimulation in vitro, prior to implantation into a human or other animal subject. In another embodiment, the stem cells are exposed to electric stimulation in situ, after implantation into the subject.

The strength of the electric field produced during electrical stimulation is preferably at least about 0.5 microvolts per centimeter. In embodiments involving administration of direct current, the current is preferably a direct current signal of at least about 0.5 microamperes. The field may be constant, or varying over time. A field that is varying over time can be a sinusoidally varying field. In one embodiment, a temporally varying capacitatively coupled field is used. In one embodiment, a sinusoidally-varying electric field is a sinusoidally-varying electric field for electrodes placed across tissue, such as a site of stem cell implantation in a human patient, for example, a human limb. Preferably, such a sinusoidally-varying electric field has a peak voltage across electrodes placed across the cells of from about 1 volt to about 10 volts, more preferably about 5 volts. In another embodiment, a sinusoidally-varying electric field is produced by electrodes placed across an in vitro culture of stem cells. Preferably, the electric field has a peak amplitude of from about 0.1 millivolt per centimeter (mV/cm) to about 100 mV/cm, more preferably about 20 mV/cm. In a preferred embodiment, the temporally varying field is sinusoidal, preferably having a frequency of from about 1,000 Hz to about 200,000 Hz, preferably about 60,000 Hz.

An electric field used herein can be produced using any suitable method and apparatus, including such methods and apparatuses known in the art. Suitable methods include exposure of stem cells to an electric field generated with the aid of a capacitatively coupling device such as a SpinalPak® (EBI, L.P., Parsippany, N.J., U.S.A.) or a DC stimulation device such as an SpF® XL IIb spinal fusion stimulator (EBI, L.P.). PEMF can be produced using any known method and apparatus, such as using a single coil or a pair of Helmholtz coils. For example, such an apparatus includes the EBI Bone Healing System® Model 1026 (EBI, L.P.).

Exposure of stem cells to PEMFs in vitro can be accomplished using methods and apparatuses known in the art for exposure of other cell types to pulsed fields, for example, as disclosed in the following literature: Binderman et al., Biochimica et Biophysica Acta 844: 273-279, 1985; Aaron et al, Journal of Bone and Mineral Research 4: 227-233, 1989; and Aaron et al., Journal of Orthopaedic Research 20: 233-240, 2002. Parameters of cell exposure to an electric stimulation field, such as, for example, pulse duration, pulse intensity, and numbers of pulses, either in vitro or in situ, can be determined by a user. In one embodiment, pulse duration of a PEMF can be from about 10 microseconds per pulse to about 2000 microseconds per pulse, and is preferably about 225 microseconds per pulse. In one embodiment, pulses are comprised in electromagnetic “bursts.” A burst can comprise from one pulse up to about two hundred pulses. Preferably, a burst comprises from about ten pulses to about thirty pulses, more preferably about twenty pulses. Bursts can be repeated while applying PEMFs to stem cells in vitro or in situ. In some embodiments, bursts can be repeated at a frequency of from about one Hertz (Hz) to about 100 Hz, preferably at a frequency of about 10 Hz to about 20 Hz, more preferably at a frequency of about 15 Hz. In addition, in some preferred embodiments, bursts can repeat at a frequency of about 1.5 Hz, or about 76 Hz. A burst can have a duration from about ten microseconds up to about 40,000 microseconds, preferably, a burst can have a duration of about 4.5 milliseconds.

In one embodiment, the present invention provides methods comprising the administration of electrical stimulation to stem cells in situ, after implantation of the stem cells into a human or other mammal subject. In one embodiment, the electrical stimulation comprises direct current electric field generated using any known device for generating a direct current electric field, such as, for example, an Osteogen™ implantable bone growth stimulator (EBI, L.P., Parsippany, N.J.). In one embodiment involving administration of direct current, the direct current electric field administered to stem cells has an intensity of from about 10 to about 200, preferably from about 20 to about 100 microamperes. Specific embodiments include those wherein the intensity is about 20, about 60, and about 100 microamperes.

Methods of Treatment:

The present invention provides methods of treating a human or other mammal subject in need thereof, using electrically stimulated stem cells. In one embodiment, such methods comprise providing an in vitro culture comprising stem cells, administering an electric stimulation to the in vitro culture, and implanting the stem cells into the subject. In another embodiment, such methods comprise implanting stem cells into the mammal subject, and administering an electric stimulation to the stem cells in situ. Administration of electrical stimulation “in situ” refers to subjecting stem cells to electrical stimulation after they have been implanted in a human or other mammal subject.

In a preferred embodiment, the present invention provides methods of treating a human or other mammal subject having a tissue defect, using electrically stimulated stem cells that are implanted at the site of the tissue defect. In one embodiment, such methods comprise providing an in vitro culture comprising stem cells, administering an electric stimulation to the in vitro culture, and implanting the stem cells at the site of the defect. In another embodiment, such methods comprise implanting stem cells into the mammal subject at the site of the defect, and administering an electric stimulation to the stem cells in situ.

As referred to herein, such “tissue defects” include any condition involving tissue which is inadequate for physiological or cosmetic purposes. Examples of such defects include those that are congenital, those that result from or are symptomatic of disease or trauma, and those that are consequent to surgical or other medical procedures. Embodiments include treatment for vascular, bone, skin, and organ tissue defects. Examples of such defects include those resulting from osteoporosis, spinal fixation procedures, hip and other joint replacement procedures, chronic wounds, myocardial infarction, fractures, sclerosis of tissues and muscles, Alzheimer's disease, and Parkinson's disease.

In one embodiment, the compositions and methods of this invention may be used to repair bone or cartilage defects. A preferred embodiment is for the treatment of bone defects. As referred to herein, such “bone defects” include any condition involving skeletal tissue which is inadequate for physiological or cosmetic purposes. Examples of such defects include those that are congenital (including birth defects), those that result from disease or trauma, and those that are consequent to surgical or other medical procedures. Examples of such defects include those resulting from bone fractures (such as hip fractures and spinal fractures), osteoporosis, spinal fixation procedures, intervertebral disk degeneration (e.g., herniation), and hip and other joint replacement procedures.

In one embodiment, stem cells are implanted in the culture medium in which they are grown. In another embodiment, stem cells are isolated from the culture medium, and implanted. In one embodiment, a biocompatible scaffold for the stem cells is implanted in the human or mammal subject at the site at which the stem cells are implanted. As referred to herein, a “scaffold” is a material that contains or supports stem cells, preferably enabling their growth at the site of implantation. A scaffold material can be an osteoconductive material. In one embodiment, the stem cells are mixed with the scaffold material prior to implantation. In other embodiments, the scaffold material is implanted before and/or after the stem cells are implanted.

Suitable scaffold materials include porous or semi-porous, natural, synthetic or semisynthetic materials. Scaffold materials include those selected from the group consisting of bone (including cortical and cancellous bone), demineralized bone, ceramics, polymers, and combinations thereof. Ceramics include any of a variety of ceramic materials known in the art for use for implanting in bone, including calcium phosphate (including tricalcium phosphate, tetracalcium phosphate, hydroxyapatite, and mixtures thereof. Polymers include collagen, gelatin, polyglycolic acid, polylactic acid, polypropylenefumarate, and copolymers or combinations thereof. Ceramics useful herein include those described in U.S. Pat. No. 6,323,146 to Pugh et al., issued Nov. 27, 2001, and U.S. Pat. No. 6,585,992 to Pugh et al., issued Jul. 1, 2003. A preferred ceramic is commercially available as ProOsteon™ from Interpore Cross International, Inc. (Irvine, Calif., U.S.A.).

Compositions:

The present invention also provides compositions comprising electrically stimulated stem cells and a biocompatible carrier. Preferably the biocompatible carrier is a scaffold material. Optionally, the compositions of this invention additionally comprise growth factors, including VEGF-1, fibroblast growth factor-2 (FGF-2), epidermal growth factor (EGF), insulin-like growth factor-1 (IGF-1), TGF-β, PDGF, IGF-I, IFG-II, EGM, and bone morphogenic protein (BMP)-2, -4, -6 and -7.

The following examples illustrate the compositions and methods of the present invention.

### EXAMPLE 1

In a method for enhancing spinal disc repair, adult autologous or allogeneic mesenchymal stem cells grown in vitro are injected into a degenerating spinal disc in a human patient. An external device providing a capacitatively coupled electric field, or a PEMF is then worn by the patient. Exposure of the implanted mesenchymal stem cells to an electric or electromagnetic field produced by the device stimulates the stem cells to proliferate and differentiate into nucleus cells and annulus disc cells and also increases extracellular matrix production by those cells, leading to disc repair.

### EXAMPLE 2

In a method for healing vertebra after posterolateral spine fusion, bone marrow-derived adult mesenchymal stem cells are mixed with osteoconductive granules comprising a calcium phosphate material such as hydroxyapatite, and implanted into a patient. An implantable direct current stimulator is placed internally in the vicinity of the graft to provide an electric field in situ to enhance bone formation. Bone healing is accelerated through this treatment.

In the above example, non invasive electrical stimulation is effected using an electric or electromagnetic field generating device to apply capacitatively coupled electric fields or PEMFs, with substantially similar results. Also, in the above example, a composition comprising a scaffold material, such as demineralized bone and/or collagen is implanted with the stem cells, with substantially similar results.

### EXAMPLE 3

In a method of this invention, a hip fracture is treated with a stem cell composition of this method. A culture system is used to expand mesenchymal stem cell numbers or generate three-dimensional constructs. In this system, mesenchymal stem cells are derived from muscle, and grown in culture dishes placed between pairs of Helmholtz coils to generate a uniform PEMF. The stem cells are then harvested, and mixed with collagen as a scaffold material. The composition is then implanted at the site of the fracture, thereby accelerating healing of the bone.

In the above example, the stem cells are grown in culture dishes placed within a capacitatively coupled electric field, with substantially similar results. Also in the above example, the stem cells are derived from bone marrow, muscle, fat, umbilical cord blood, or placenta, with substantially similar results. Also in the above example, collagen are replaced with polyglycolic acid or polylactic acid, with substantially similar results.

### EXAMPLE 4

In a method of this invention, the differentiation of mesenchymal stem cells is enhanced with PEMF. Human mesenchymal stem cells are plated in culture dishes such as, for example, 10 cm² culture dishes, and the non-differentiating cultures are grown to near confluence. The cells in the dishes are then stimulated to undergo osteoblast differentiation in the presence or absence of PEMFs. Samples are taken at different times throughout the differentiation process and examined. Day of plating is designated as day-2. At day 0 (2 days later), cells are stimulated down the osteoblast differentiation pathway. Osteoblast differentiation (to mineralization in vitro) is induced with osteoblast medium (Mesenchymal Stem Cell Growth Medium/10% Fetal Bovine Serum/0.1 μM dexamethasone/50 μM ascorbate/10 mM β-glycerophosphate/50 ng/ml BMP-4). Cell numbers and extracts are collected at days 0, 1, 2, 6, 9, 12, 14, 21, and 28 following mineralization stimulus. Some cells are stained for mineralized state, others are used for Western blot, RNA extraction, osteocalcin assays, and alkaline phosphatase assays. The stem cells subjected to PEMF show increased proliferation compared to control stem cells, as evidenced by increased incorporation of ³²P dCTP, as well as increased differentiation of osteoblasts, as evidenced by increased amounts of osteocalcin and alkaline phosphatase, as well as various osteocalcin mRNAs detected using Northern blot analysis.

### EXAMPLE 5

The growth of stem cells is enhanced in a method of this invention. Stem cell cultures of equal cell density are plated in IMDM+10% FBS+1% L-glutamine+1×penicillin/streptomycin+4 ng/ml FGF-2. Cells are seeded in a 6-well plate, at 3600 cells/cm², for 12 days. Electromagnetic fields are applied for 8 hours per day. Media is changed on days 4, 7, 9 and 11. The electromagnetic field-treated cell cultures show substantially increased cell density compared to control cells.

The examples and other embodiments described herein are exemplary and not intended to be limiting in describing the full scope of compositions and methods of this invention. Equivalent changes, modifications and variations of specific embodiments, materials, compositions and methods may be made within the scope of the present invention, with substantially similar results.

1. A method of modulating mesenchymal stem cell activity, comprising administering electric stimulation to the mesenchymal stem cells in vitro.

2. A method according to claim 1, wherein modulating a mesenchymal stem cell activity comprises increasing the proliferation rate of the mesenchymal stem cells.

3. A method according to claim 1, wherein modulating an activity of a mesenchymal stem cell comprises promoting differentiation of the mesenchymal stem cells.

4. A method according to claim 3, wherein promoting differentiation of the mesenchymal stem cells comprises promoting the differentiation of mesenchymal stem cells into cells selected from the group consisting of bone, cartilage, vasculature and blood cells.

5. A method according to claim 4, wherein promoting differentiation of the mesenchymal stem cells comprises promoting the differentiation of mesenchymal stem cells into cells selected from the group consisting of bone cells and cartilage cells.

6. A method according to claim 1, wherein the mesenchymal stem cells are human mesenchymal stem cells.

7. A method according to claim 6, wherein the mesenchymal stem cells are selected from the group consisting of umbilical cord stem cells, muscle stem cells, placental stem cells, fat stem cells, bone marrow stem cells, and synovium stem cells.

8. A method according to claim 8, wherein the umbilical cord stem cells are umbilical cord blood stem cells.

9. A method according to claim 1, wherein the electric stimulation comprises a direct current electric field.

10. A method according to claim 9, wherein the direct current electric field comprises a direct current signal of from about ten microamperes to about two hundred microamperes.

11. A method according to claim 10, wherein the direct current electric field comprises a direct current signal of from about twenty microamperes to about one hundred microamperes.

12. A method according to claim 1, wherein the electric stimulation comprises a capacitatively coupled electric field.

13. A method according to claim 12, wherein the capacitatively coupled electric field is a sinusoidally varying electric field.

14. A method according to claim 13, wherein the sinusoidally-varying electric field has a peak voltage across electrodes across the cells of from about 1 volt to about 10 volts.

15. A method according to claim 13, wherein the sinusoidally-varying electric field is a field across an in vitro culture of stem cells having a peak amplitude of from about 0.1 mV/cm to about 100 mV/cm.

16. A method according to claim 13, wherein the sinusoidally varying current electric field has a frequency of about 1,000 Hz to about 200,000 Hz.

17. A method according to claim 16, wherein the sinusoidally varying current electric field has a frequency of about 60,000 Hz.

18. A method according to claim 1, wherein administering electric stimulation to the mesenchymal stem cells in vitro comprises administering a pulsed electromagnetic field to the mesenchymal stem cells in vitro.

19. A method according to claim 18, wherein administering a pulsed electromagnetic field to the mesenchymal stem cells in vitro comprises applying a pulsed electromagnetic field using paired Helmholtz coils.

20. A method according to claim 19, wherein the administering a pulsed electromagnetic field to the mesenchymal stem cells comprises administering a plurality of electromagnetic pulses to the mesenchymal stem cells, the plurality of electromagnetic pulses comprising electromagnetic pulses of a duration about 10 microseconds per pulse to about 2000 microseconds per pulse.

21. A method according to claim 20, wherein a plurality of electromagnetic pulses comprises a burst of from one pulse to about two hundred pulses.

22. A method according to claim 21, wherein a burst repeats at a frequency of from about 1 Hz to about 100 Hz.

23. A method according to claim 22, wherein each burst comprises a duration of about 2 milliseconds to about 40 milliseconds.

24. A method of treatment of a human or other mammal subject in need thereof, the method comprising providing an in vitro culture comprising mesenchymal stem cells, administering an electric stimulation to the in vitro culture, and implanting the mesenchymal stem cells into the subject.

25. A method according to claim 24, wherein the treatment is a treatment for a tissue defect, injury, disorder or disease.

26. A method according to claim 25, wherein the tissue defect, injury, disorder or disease is a bone defect, injury, disorder or disease.

27. A method according to claim 24, wherein the implanting the mesenchymal stem cells into the mammal comprises implanting the mesenchymal stem cells to a site selected from the group consisting of a site of bone disease, fracture, wound, injury, birth defect, spinal fusion, defective cartilage, a site of an orthopedic implant, a degenerated or herniated intervertebral disk, and a site of intervertebral disk replacement.

28. A method according to claim 24, wherein modulating a mesenchymal stem cell activity comprises increasing the proliferation rate of the mesenchymal stem cells.

29. A method according to claim 24, wherein modulating an activity of a mesenchymal stem cell comprises promoting differentiation of the mesenchymal stem cells.

30. A method according to claim 29 wherein the promoting differentiation of the mesenchymal stem cells comprises promoting the differentiation of mesenchymal stem cells into cells selected from the group consisting of bone cells and cartilage cells.

31. A method according to claim 24, wherein the mesenchymal stem cells are human mesenchymal stem cells.

32. A method according to claim 24, wherein the mesenchymal stem cells are selected from the group consisting of umbilical cord stem cells, muscle stem cells, placental stem cells, fat stem cells, bone marrow stem cells, and synovium stem cells.

33. A method according to claim 24, wherein the mesenchymal stem cells are autologous mesenchymal stem cells.

34. A method according to claim 24, wherein the mesenchymal stem cells are allogeneic mesenchymal stem cells.

35. A method according to claim 24, wherein the direct current electric field comprises a direct current signal of from about ten microamperes to about two hundred microamperes.

36. A method according to claim 24, wherein the capacitatively coupled electric field is a sinusoidally varying current electric field.

37. A method according to claim 36, wherein the sinusoidally-varying electric field has a peak voltage across electrodes across the cells of from about 1 volt to about 10 volts.

38. A method according to claim 36, wherein the sinusoidally-varying electric field is a sinusoidally-varying electric field across an in vitro culture of stem cells having a peak amplitude of from about 0.1 mV/cm to about 100 mV/cm.

39. A method according to claim 36, wherein the sinusoidally varying current electric field has a frequency of about 1,000 Hz to about 200,000 Hz.

40. A method according to claim 24, wherein the administering electric stimulation to the mesenchymal stem cells in vitro comprises administering a pulsed electromagnetic field.

41. A method according to claim 40, wherein the administering a pulsed electromagnetic field to the mesenchymal stem cells in vitro comprises applying a pulsed electromagnetic field using paired Helmholtz coils.

42. A method according to claim 41, wherein administering a pulsed electromagnetic field to the mesenchymal stem cells comprises administering a plurality of electromagnetic pulses to the mesenchymal stem cells, the plurality of electromagnetic pulses comprising electromagnetic pulses of a duration about 10 microseconds per pulse to about 2000 microseconds per pulse.

43. A method according to claim 24, further comprising implanting osteoconductive granules at the site where said stem cells are implanted.

44. A method according to claim 24, further comprising implanting a scaffold material at the site where said stem cells are implanted.

45. A method of treatment of a human or other mammal subject in need thereof, comprising providing an in vitro culture comprising mesenchymal stem cells, implanting the mesenchymal stem cells into the mammal subject, and administering an electric stimulation to the mesenchymal stem cells in situ.

46. A method according to claim 45, wherein the treatment is a treatment for a bone defect, injury, disorder or disease.

47. A method according to claim 45 wherein the implanting the mesenchymal stem cells into the mammal comprises implanting the mesenchymal stem cells to a site selected from the group consisting of a site of bone disease, fracture, wound, injury, birth defect, spinal fusion, defective cartilage, a site of an orthopedic implant, a degenerated or herniated intervertebral disk, and a site of intervertebral disk replacement.

48. A method according to claim 45, wherein the mesenchymal stem cells are human mesenchymal stem cells.

49. A method according to claim 45, wherein the mesenchymal stem cells are selected from the group consisting of umbilical cord stem cells, muscle stem cells, placental stem cells, fat stem cells, bone marrow stem cells, and synovium stem cells.

50. A method according to claim 45, wherein the direct current electric field comprises a direct current signal of from about ten microamperes to about two hundred microamperes.

51. A method according to claim 45, wherein the capacitatively coupled electric field is a sinusoidally varying current electric field.

52. A method according to claim 51, wherein the sinusoidally-varying electric field has a peak voltage across electrodes across the cells of from about 1 volt to about 10 volts.

53. A method according to claim 51, wherein the sinusoidally-varying electric field is a sinusoidally-varying electric field across an in vitro culture of stem cells having a peak amplitude of from about 0.1 mV/cm to about 100 mV/cm.

54. A method according to claim 51, wherein the sinusoidally varying current electric field has a frequency of about 1,000 Hz to about 200,000 Hz.

55. A method according to claim 45, wherein the administering electric stimulation to the mesenchymal stem cells in vitro comprises administering a pulsed electromagnetic field.

56. A method according to claim 55, wherein administering a pulsed electromagnetic field to the mesenchymal stem cells comprises administering a plurality of electromagnetic pulses to the mesenchymal stem cells, the plurality of electromagnetic pulses comprising electromagnetic pulses of a duration about 10 microseconds per pulse to about 2000 microseconds per pulse.

57. A method according to claim 45, further comprising adding osteoconductive granules at the site where said stem cells are implanted.

58. A method according to claim 45, further comprising implanting a scaffold material at the site where said stem cells are implanted.
